(19) United States
(12) Patent Application Publication
(10) Pub. No.: US 2005/0249749 A1
(43) Pub. Date: Nov. 10, 2005

(54) B-cell epitopes in the N-terminal repeated peptides of Anaplasma marginale major surface protein 1a, pharmaceutical compositions and methods of use

(76) Inventors: Jose de Jesus de la Fuente, Stillwater, OK (US); Katherine M. Kocan, Perkins, OK (US); Edmour F. Blouin, Perkins, OK (US); Jose Carlos Garcia-Garcia, Baltimore, MD (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 11/089,764

(22) Filed: Mar. 25, 2005

Related U.S. Application Data
(63) Continuation-in-part of application No. 10/285,319, filed on Oct. 31, 2002.
Application No. 10/285,319 is a continuation-in-part of application No. 10/002,636, filed on Oct. 26, 2001, now Pat. No. 6,939,549.
(60) Provisional application No. 60/244,333, filed on Oct. 30, 2000.

(51) Int. Cl. G01N 33/554; G01N 33/569; C07H 21/04; A61K 39/02; C12N 1/21; C07K 14/20; C12N 15/74

(52) U.S. Cl. 424/190.1; 435/69.3; 435/252.3; 435/471; 530/350; 536/23.7

(57) ABSTRACT

B-cell epitopes in the N-terminal repeated peptides of Anaplasma marginale major surface protein (MSP)1a are characterized. Pharmaceutical compositions including such epitopes, and methods useful to induce or enhance an immune response against infection by ehrlichial parasites of the species Anaplasma marginale, are provided.

### CROSS REFERENCE TO RELATED APPLICATIONS

This application is a continuation-in part of prior filed, copending U.S. patent application Ser. No. 10/285,319, filed Oct. 31, 2002, which is a continuation-in-part of U.S. patent application Ser. No. 10/002,636, filed Oct. 26, 2001, which claims the benefit of U.S. provisional patent application Ser. No. 60/244,333, filed Oct. 30, 2000, all of which are hereby incorporated by reference.

### BACKGROUND OF THE INVENTION

1. Technical Field

The present invention relates to the discovery of B-cell epitopes in the N-terminal repeated peptides of Anaplasma marginale major surface protein (MSP)1a and encompasses such epitopes, pharmaceutical compositions including such epitopes, and methods useful to induce or enhance an immune response against infection by ehrlichial parasites of the species Anaplasma marginale.

2. Background

As used herein, the following abbreviations have the ascribed meaning: BPL, β-propiolactone; MHC, major histocompatibility complex; MSP, major surface protein; PCV, packed cell volume; PPE, percent parasitized erythrocytes; rMSP1a, recombinant major surface protein 1a; TBS, Tris-buffered saline.

Bovine anaplasmosis is a tick-borne disease of cattle caused by the obligate intraerythrocytic rickettsia Anaplasma marginale (Rickettsiales: Anaplasmataceae). During the course of infection the number of infected erythrocytes increases geometrically and removal of these infected cells by phagocytosis results in severe anemia, weight loss, abortion, and often death (Kuttler, 1984). Cattle that recover from acute infection remain persistently infected with A. marginale, are protected from homologous challenge (Kuttler, 1984), and serve as reservoirs for mechanical and biological transmission of A. marginale (as reviewed by Dikmans, 1950 and Ewing, 1981).

Five major surface proteins have been identified on erythrocytic and tick stages of A. marginale. Four of these MSPs, designated MSP1, MSP2, MSP3 and MSP4, were identified initially using neutralizing polyclonal antibodies (Palmer and McGuire, 1984), and subsequently MSP5 was identified (Visser et al., 1992). All of these MSPs were shown to be structurally conserved in A. marginale derived from bovine erythrocytes and tick cells (Barbet et al., 1999).

The MSP1 complex is composed of two covalently linked unrelated polypeptides, MSP1a and MSP1b, which have been shown to be involved in adhesion of A. marginale to host cells. MSP1a is an adhesin for both bovine erythrocytes and tick cells, whereas MSP1b is an adhesin only for bovine erythrocytes (McGarey & Allred, 1994; McGarey et al., 1994, de la Fuente et al., 2001 a). MSP1a has also been shown to be involved in infection and transmission of A. marginale by ticks (de la Fuente et al., 2001a; Blouin et al., 2003). The molecular size of MSP1a varies among isolates of A. marginale due to a different number of tandemly repeated peptides in the N-terminal region of MSP1a. These repeated peptides are surface-exposed, contain a neutralization-sensitive epitope (Palmer et al., 1987; de la Fuente et al., 2001c), and were shown to be necessary and sufficient for adhesion of A. marginale to host cells (de la Fuente et al., 2003b).

Methods for the control of A. marginale have included vector control, vaccination and the use of antibiotics (reviewed by Kocan et al., 2003). Vaccination induces protective immunity in cattle, however anaplasmosis vaccines (live, attenuated or killed whole-organism) using erythrocyte-derived antigen may have the disadvantages of being contaminated with erythrocyte stroma, bear the risk of transmitting other pathogens, and these vaccines are expensive to produce because they require the use of cattle as a source of infected erythrocytes. A. marginale derived from cultured tick cell lines provides an alternate source of antigen that overcomes these drawbacks and the cell culture derived A. marginale has recently been shown to induce a protective immune response in cattle (reviewed by Kocan et al., 2003). Other approaches tested for the immunological control of anaplasmosis are based on vaccination with native or recombinant A. marginale surface proteins or naked DNA (Palmer et al., 1986; 1988; 1989; Tebele et al., 1991; McGuire et al., 1994; de la Fuente et al., 2003a; reviewed by Kocan et al., 2003). A differential antibody response to MSP1a and MSP1b was observed in cattle immunized with A. marginale derived from bovine erythrocytes or cultured tick cells (Kocan et al., 2001; de la Fuente et al., 2002a; Garcia-Garcia et al., 2003). Cattle immunized with erythrocyte-derived A. marginale developed a preferential antibody response to MSP1a, whereas cattle immunized with tick cell culture-derived A. marginale developed a stronger anti-MSP1b response. This difference was found to result from the up-regulated expression of MSP1a by A. marginale in bovine erythrocytes and low-level expression of MSP1a by organisms in tick cells (Garcia-Garcia et al., 2003).

Clearance of A. marginale infection by the bovine immune system is mediated by the development of both a humoral immune response against surface-exposed epitopes and a CD4⁺ T-cell-mediated response (reviewed by Palmer et al., 1999). Antibodies against A. marginale major surface proteins are involved in three main mechanisms of protection against A. marginale infection, including neutralization due to the direct action of antibodies, antibody-dependent cellular cytotoxicity by MHC non-restricted lymphocytes and macrophage phagocytosis mediated by opsonizing antibodies (reviewed by Palmer et al., 1999). Protective immunity against A. marginale can be stimulated by vaccination with live or killed organisms, initial body membranes, purified native or recombinant outer membrane proteins, or DNA encoding for A. marginale MSPs (Palmer et al., 1989; Montenegro-James et al., 1991; Tebele et al., 1991; Arulkanthan et al., 1999; Kocan et al., 2001; de la Fuente et al., 2002a). Protection against A. marginale infection has been shown to correlate with the level of antibodies specific for A. marginale MSPs (Tebele et al., 1991).

Antibodies to MSP1a have been shown to inhibit A. marginale infection of bovine erythrocytes (Palmer et al., 1986) and cultured tick cells (Blouin et al., 2003) and to decrease infection of salivary glands of ticks fed on cattle with antibodies to MSP1a (de la Fuente et al., 2003a). Polyclonal antibodies to MSP1a have also been shown to inhibit adhesion to bovine erythrocytes mediated by MSP1a (McGarey et al., 1994). MSP1a contains CD4⁺ T-lymphocyte epitopes in the conserved C-terminal region (Brown et al., 2001; 2002), but bovine B-cell epitopes of MSP1a have not been described.

### SUMMARY OF THE INVENTION

In connection with the present invention, the MSP1a antibody response of cattle was characterized using several immunogens, including recombinant MSP1a (rMSP1a) protein, erythrocyte- or tick cell culture-derived A. marginale, or a combination of tick cell culture-derived A. marginale and rMSP1a. The MSP1a antibody response to all these immunogens was directed primarily against the N-terminal region of MSP1a, whereas low antibody levels were detected against the C-terminal portion. Linear B-cell epitopes of MSP1a were mapped using synthetic peptides representing the entire sequence of the protein that were prepared by SPOT synthesis technology. Only two peptides in the N-terminal repeats were recognized by sera from immunized cattle. These peptides shared the sequence SSAGGQQQESS (SEQ. ID NO: 77), which is likely to contain the linear B-cell epitope that was recognized by the pools of bovine sera. The average differential of antibody titers against MSP1a minus those against MSP1b correlated with lower percent reductions in PCV. A preferential antibody response to MSP1a was observed in cattle immunized with erythrocyte-derived, cell culture-derived plus rMSP1a or rMSP1a alone, and the percent reduction PCV was significantly lower in these cattle as compared with the other immunization groups. This insight into the bovine antibody response against A. marginale and the role of MSP I a in protection of cattle against A. marginale infection renders more effective vaccine strategies for control of anaplasmosis.

In one aspect of the present invention there are thus provided novel peptides comprising sequences containing B-cell epitopes of MSP1 a purified surface protein antigen of A. marginale.

In another aspect of the present invention there is provided a pharmaceutical composition for inducing or enhancing an immune response in a ruminant against A. marginale, the composition comprising one or more discrete B-cell epitopes of MSP1a purified surface protein antigen of A. marginale, alone or in combination with other antigenic components, wherein the composition further comprises a pharmaceutically acceptable carrier or diluent.

In still another aspect of the present invention there is provided a method for inducing or enhancing an immune response in a ruminant to provide immune protection against infection by A. marginale, the method comprising administering to said ruminant an effective amount of the inventive pharmaceutical composition.

A better understanding of the present invention, its several aspects, and its advantages will become apparent to those skilled in the art from the following detailed description, taken in conjunction with the attached drawings, wherein there is shown and described the preferred embodiment of the invention, simply by way of illustration of the best mode contemplated for carrying out the invention.

### BRIEF DESCRIPTION OF THE DRAWINGS

FIG. 1 is a graph illustrating the antibody response against A. marginale MSP5 in immunized and control cattle. Eight groups of five animals each were immunized with MSP1 complex (MSP1), rMSP1a, rMSP1b, rMSP1a plus rMSP1b, cell culture-derived A. marginale with (rMSP1a+CCDA) or without (CCDA) rMSP1a, erythrocyte-derived A. marginale (EDA), or adjuvant alone (Saline) at 1, 4 and 7 weeks of the experiment (arrows). Antibody levels against MSP5 were measured by competitive ELISA. All cattle were challenge exposed (indicated by star) at week 9 with 10⁹ A. marginale.

FIG. 2 is a graph illustrating the antibody response against A. marginale MSP1a and MSP1b in immunized and control cattle. Serum samples were collected at the peak antibody response (week 9), approximately two weeks after the last immunization and prior to challenge. The antibody levels against MSP1a and MSP1b were measured by ELISA. Bars represent the geometric mean titer (mean±S.D.) of the five cattle of each group.

FIG. 3 is a graph illustrating the antibody response against A. marginale MSP1a and its N-terminal repeated peptides and C-terminal regions at the peak antibody response (week 9). Recombinant MSP1a, MSP1a repeats and C-terminal region were expressed in E. coli and used for coating ELISA plates. Bars represent the geometric mean titer (mean±S.D.) of the five cattle per group.

FIG. 4 is a chart summarizing the linear B-cell epitope mapping of MSP1a. Pools of sera from cattle (Bov) and mice immunized with rMSP1a, cell culture-derived A. marginale with (rMSP1a+CCDA) or without (CCDA) rMSP1a or erythrocyte-derived A. marginale (EDA) were allowed to react with peptides synthesized using SPOTs technology. The amino acid sequences of the overlapping synthetic peptides that span the whole MSP1a protein from the Oklahoma isolate of A. marginale are indicated. Sera from rabbits (Rab) immunized with rMSP1a or a synthetic peptide, R2FL, that models the MSP1a repeats were also analyzed. Mouse monoclonal antibody 15D₂ was used as a positive control. Black boxes represent recognition of the peptide by the corresponding antibodies. The location of each peptide in the predicted topology of the MSP1a protein is indicated in the left column. Residues in the inner (In) or outer (Out) side of the membrane (M) are indicated. Transmembrane helices (in boldface) were predicted using the TMHMM2 algorithm.

FIG. 5 is a graph illustrating the effect of antibodies specific for MSP1a and MSP1b on protection against A. marginale infection. The group mean percent reduction PCV was correlated with the group mean differential antibody titers (MSP1a minus MSP1b). Percent reduction PCV was calculated from the lowest PCV after challenge and the average PCV prior to challenge. The trendline was fitted to a cubic (third degree) polynomial equation using Microsoft Excel. Immunization groups are indicated.

### DETAILED DESCRIPTION OF THE PREFERRED EMBODIMENT

Before explaining the present invention in detail, it is important to understand that the invention is not limited in its application to the details of the embodiments and steps described herein. The invention is capable of other embodiments and of being practiced or carried out in a variety of ways. It is to be understood that the phraseology and terminology employed herein is for the purpose of description and not of limitation.

In accordance with the present invention there is provided B-cell epitopes derived from MSP1a useful as antigenic components of a pharmaceutical composition. They may also be utilized in combination with other antigenic components. The invention further encompasses the vaccination of ruminants using the B-cell epitopes.

The present invention will be further understood with reference to the following non-limiting description of experimental results and examples.

Materials and Methods

Anaplasma marginale Isolates

The Oklahoma isolate of A. marginale was used for the cattle immunization studies and the Virginia isolate was used for challenge exposure of immunized cattle. Both of these isolates have been shown to be tick transmissible and have been propagated in cultured tick cells in our laboratory (Munderloh et al., 1996; Blouin et al., 2000; de la Fuente et al., 2001b; 2002b).

Bovine Erythrocyte-Derived A. marginale

A susceptible splenectomized 3-month-old calf (PA479) was experimentally infected with blood stabilate of the Oklahoma isolate of A. marginale that was collected from calf PA407 with a percent parasitized erythrocytes (PPE) of 10% (Blouin et al., 2000). Calf PA481 was infected with the Virginia isolate of A. marginale by intravenous inoculation of blood stabilate from PA433 (de la Fuente et al., 2002b) with a PPE of 12.2% and a packed cell volume (PCV) of 28.5%. The calves were maintained by the OSU Laboratory Animal Resources according to the Institutional Care and Use of Animals Committee guidelines. Infection of the calves was monitored by examination of stained blood smears. Bovine erythrocytes were collected from calf PA479 at a PPE of 32.2%, washed three times in PBS, each time removing the buffy coat, and stored at −70° C. Infected erythrocytes were thawed and A. marginale antigen quantified by use of an MSP5 antigen detection ELISA (Saliki et al., 1998), inactivated with β-propiolactone (BPL), and doses of approximately 2×10¹⁰ A. marginale were prepared for immunization of cattle (Table 1). Blood from calf PA481 was collected during ascending parasitemia and used for challenge-exposure of vaccinated cattle.

Tick Cell Culture-Derived A. marginale

A. marginale was propagated in the tick cell line, IDE8 (ATCC CRL 11973), derived originally from Ixodes scapularis embryos as described previously (Munderloh et al., 1996; Blouin et al., 2000). Monolayers of IDE8 cells were inoculated with a blood stabilate of the Oklahoma isolate of A. marginale that was retrieved from liquid nitrogen. Approximately 10 days post-inoculation terminal cultures with >90% infected cells were harvested by centrifugation, resuspended in PBS and stored at −70° C. until used for antigen preparation (Table 1). A. marginale antigen was quantified by use of an MSP5-specific antigen detection ELISA (Saliki et al., 1998) and then inactivated with BPL. Antigen doses were prepared that contained approximately 2×10¹⁰ A. marginale.

Recombinant MSP1 Proteins, Expression and Purification

The msp1α and msp1β₁ genes of the Oklahoma isolate of A. marginale, encoding for MSP1a and MSP1b, respectively, were cloned by PCR, fused to the FLAG peptide and expressed in E. coli as reported previously (de la Fuente et al., 2001a). Recombinant E. coli cells expressing MSP1a and MSP1b proteins were collected and disrupted by sonication. The membrane fractions containing the recombinant proteins were used for preparation of immunogens (100 μg/dose) for cattle vaccination (Table 1) and for the purification of the recombinant proteins. The recombinant MSP1a and MSP1b proteins were extracted with 0.1% Triton X-100 in TBS and purified by FLAG-affinity chromatography (Sigma, USA) following the manufacturer's instructions. Affinity-purified recombinant proteins were used as ELISA coating antigen for the serological evaluation of vaccinated cattle. Expression and purification of the recombinant proteins was confirmed by SDS-PAGE (Laemmli, 1970) and immunoblotting. A protein complex mimicking the native MSP1 complex was obtained in vitro by diluting equal amounts of rMSP1a and rMSP1b proteins in 6 M guanidine hydrochloride, 5 mM DTT and dialyzing against a 6 M urea solution that was slowly diluted with PBS. After 48 hours the sample was dialyzed against PBS for an extra 12 hours. This in vitro obtained MSP1 complex was used in the cattle vaccination experiment (Table 1).

Construction, Expression and Purification of msp1α Mutants

Two msp1α mutants were obtained as described by de la Fuente et al. (2003b). One mutant (pF1ARO5) contained only the sequence encoding for the hydrophilic N-terminal region of the MSP1a protein that contains the tandem repeats, while the second mutant (pAFOR1) contained only the sequence encoding for the conserved C-terminal region of MSP1a. These mutants were obtained by PCR using the Oklahoma isolate msp1α gene. The mutant proteins were then expressed in E. coli purified as described above for rMSP1a and rMSP1b and used to coat ELISA plates for evaluation of the antibody response by immunized cattle.

SDS-Polyacrylamide Gel Electrophoresis and Immunoblotting

Protein samples were loaded on 10% polyacrylamide gels that were stained with Coomassie Brilliant Blue or transferred to nitrocellulose membranes. Membranes were blocked with 5% skim milk for 60 min at room temperature. Western blot analysis was performed using monoclonal antibodies ANA15D₂ (VMRD, USA) specific for the N-terminal repeats of MSP1a, anti-FLAG M2 (Sigma, USA) for detection of recombinant fusion proteins or MSP1b-monospecific rabbit serum for detection of MSP1b. After washing with TBS, the membranes were incubated with 1:10,000 goat anti-mouse IgG or goat anti-rabbit IgG alkaline phosphatase conjugate (KPL, USA).The membranes were washed again and the color was developed using BCIP/NBT alkaline phosphatase substrate (Sigma, USA).

Cattle Immunization and Challenge

Forty Holstein cattle, 12 to 24-month-old, were randomly distributed into eight groups of five animals each (Table 1). Cattle were immunized at weeks 0, 4 and 7 with a 5 ml dose of immunogen containing the antigen(s) in an oil-based adjuvant (Adjuvant XtendIII®, Novartis Animal Vaccines Inc., USA). Cattle in an additional group were immunized with saline and adjuvant to serve as controls. Cattle were challenge-exposed two weeks after the last immunization (week 9) by intravenous administration of 1.7 ml blood from calf PA481 with approximately 10⁹ infected erythrocytes of the Virginia isolate of A. marginale. Blood samples in EDTA-treated vacutainers were collected from the immunized and control cattle twice a week and then daily after detection of A. marginale infected erythrocytes for determination of the parasitemia and PCV. Protection against A. marginale infection was expressed as the percent reduction PCV calculated from the lowest PCV after challenge with respect to the initial PCV.

Production of Antibodies in Mice and Rabbits

Four groups of Balb/c mice were immunized subcutaneously at weeks 0 and 2 with 5 μg rMSP1a antigen, tick cell culture derived A. marginale, erythrocyte-derived A. marginale or cell culture-derived A. marginale supplemented with rMSP1a (Table 1). Serum samples were collected two weeks after the second immunization (week 4) and used for the analysis of B-cell epitopes. Monoclonal antibody ANA15D₂ (VMRD, USA), known to react with the linear neutralization-sensitive epitope (Q/E)ASTSS (SEQ. ID NO: 78) of the MSP1a repeated peptides (Palmer et al., 1987; Allred et al., 1990), was used as a control in the B-cell epitope mapping experiment. A New Zealand White rabbit was immunized subcutaneously two times (weeks 0, 4) with approximately 50 μg denatured rMSP1a antigen extracted from an SDS-PAGE gel. A blood sample was collected at week 6 and the sera were stored at −70° C. until used for the epitope mapping studies. Antiserum prepared previously in a rabbit immunized with a synthetic peptide (R2FL) that mimics the MSP1a repeats (de la Fuente et al., 2003b) was also used in this study.

Serologic Evaluation of Immunized Cattle

The antibody response against A. marginale MSPs in immunized and control cattle was analyzed by ELISA. Antibody levels to A. marginale MSP5 were determined by use of an A. marginale specific competitive ELISA (Saliki et al., 1998). Antibody levels to A. marginale MSP1a, MSP1b, and MSP1a mutants were detected by indirect ELISA (Garcia-Garcia et al., 2003). Briefly, purified recombinant MSP1a, MSP1b, MSP1a N-terminal repeats mutant and MSP1a C-terminal region mutant were used to coat ELISA plates for 3 hours at 37° C. The coated plates were blocked with 2% skim milk overnight at 4° C. Sera were serially diluted 1:2 from a 1:125 initial dilution. The plates were incubated with the diluted sera for 2 hours at 37° C. and then incubated with 1:2000 goat anti-bovine IgG-HRP conjugate (KPL, USA) for 1 hour at 37° C. The color reaction was developed with TMB (Sigma, USA) and the OD_{450 nm} was determined. Antibody titers were expressed as the maximum dilution of the serum that yielded an OD value at least twice as high as the negative control serum. Antisera with antibody levels not detectable at the lowest dilution (1:125) were assigned a titer of 10 for the statistical analysis. Geometric mean titers were calculated for each experimental group.

Linear B-Cell Epitope Mapping

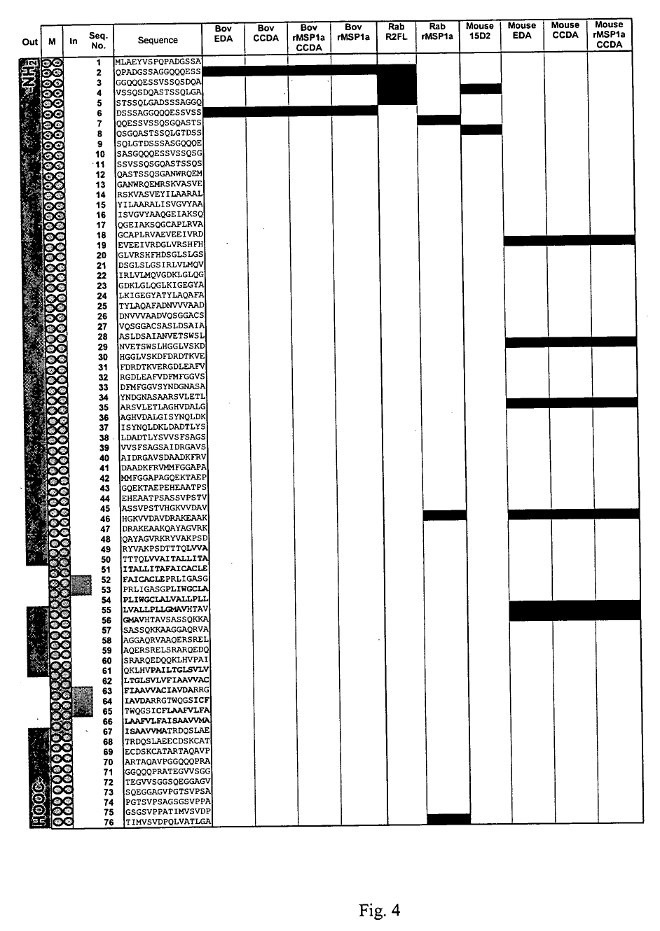

Equal volumes of serum samples collected at the peak antibody response from cattle or mice in each immunization group were pooled for mapping of linear B-cell epitopes. Seventy six overlapping 16-mer peptides (FIG. 4, SEQ. ID NOS: 1-76) covering the entire sequence of the Oklahoma isolate MSP1a were simultaneously synthesized on a cellulose membrane using SPOT synthesis technology (Sigma Genosys, USA). Before each use, the membrane was blocked with 5% skim milk in TBST for 1 h at room temperature. The membrane was then incubated for 1 h with a 1:200 dilution of pooled serum samples from groups of cattle or mice immunized with erythrocyte-derived A. marginale antigen, tick cell culture-derived A. marginale, rMSP1a protein or tick cell culture A. marginale antigen supplemented with rMSP1a. Sera from rabbits immunized with synthetic peptide R2FL that models the MSP1a N-terminal peptides (de la Fuente et al., 2003b) or with rMSP1a protein were also assayed. Monoclonal antibody ANA15D₂ (VMRD, USA) was used as a positive control. After washing with TBST, the membrane was incubated for 1 h with a 1:250,000 dilution of goat anti-bovine IgG, anti-rabbit IgG or anti-mouse IgG horseradish peroxidase conjugate (KPL, USA). The membrane was washed five times with TBST, incubated with SuperSignal® West Pico peroxidase substrate (Pierce, USA) for 5 min and exposed to X-ray film for 1 min. The membrane was regenerated to remove bound antibodies by incubating with Restore™ Western Blot Stripping buffer (Pierce, USA) for 15 minutes at room temperature. The membrane was tested for complete removal of antibodies by being reincubated between assays with horseradish peroxidase conjugate and substrate and then exposed to film.

Protein Sequence and Prediction of Protein Topology

The amino acid sequence of the MSP1a protein from the Oklahoma isolate of A. marginale were obtained from GenBank, accession number AY010247, and used for the design of the peptides synthesized for the mapping of B-cell epitopes. Protein topology was predicted using the TMHMM2 algorithm for the prediction of transmembrane helices (Krogh et al., 2001).

Statistical Analysis

The antibody titers among immunization groups were compared using analysis of variance and a Student's t-test with the Bonferroni correction for multiple comparisons. The percent reduction PCV in cattle with a preferential antibody response to MSP1a was compared to that of cattle with a preferential antibody response to MSP1b using a Student's t-test with the Bonferroni correction. A correlation analysis was performed using Microsoft Excel to study the correlation of antibody titers to MSP1a or MSP1b and the percent reduction PCV, an indicator of anemia and thus clinical disease. The group mean differential of the antibody titers against MSP1a minus the MSP1b antibody titers was also included in the correlation analysis.

Results

Immunization of Cattle and Antibody Response to the A. marginale MSPs.

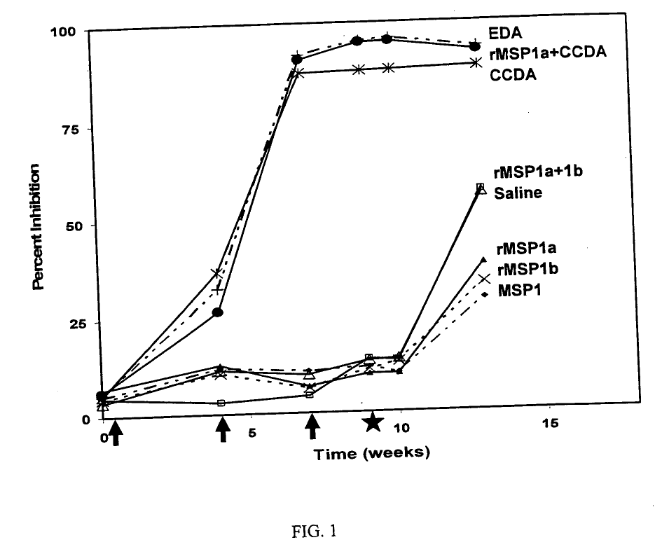

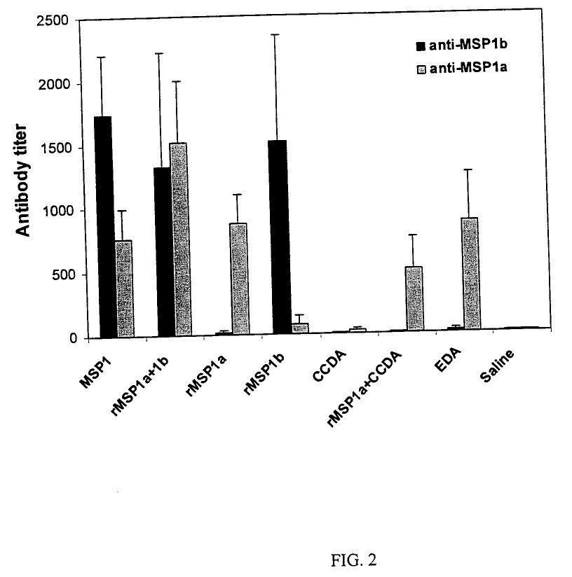

To study the MSP antibody response of immunized and control cattle, the antibody titers against MSP5, MSP1a, MSP1b, and MSP1a mutants were determined by ELISA. All cattle immunized with immunogens that contained erythrocyte- or tick cell culture-derived A. marginale seroconverted to MSP5, the surface protein used to normalize the amount of A. marginale antigen contained in the vaccine preparations. The level of MSP5 specific antibodies peaked at weeks 9-10, approximately two weeks after the last immunization (FIG. 1). Although the peak antibody levels were close to saturation of the assay, the peak MSP5 antibody levels in cattle immunized with tick cell culture-derived A. marginale were lower than that of cattle immunized with erythrocyte-derived A. marginale (FIG. 1), probably due to an overestimation of the amount of cell culture derived antigen included in the vaccine preparations. Sera from cattle that received recombinant proteins or adjuvant alone were negative for antibodies against MSP5 from weeks 0 to 10, but developed an antibody response to MSP5 after challenge-exposure with A. marginale infected erythrocytes on week 9 (FIG. 1). This boost in the antibody response was not observed in cattle vaccinated with erythrocyte- or tick cell culture-derived A. marginale, which may indicate that either these animals were protected against challenge or that the antibody levels were already high enough to detect small variations in antibody levels using a competitive ELISA. Cattle that were immunized with MSP1, rMSP1a, rMSP1b, or rMSP1a and rMSP1b developed a strong antibody response against these respective proteins (FIG. 2). The antibody response of cattle immunized with erythrocyte-derived A. marginale was predominantly against MSP1a (FIG. 2), consistent with previous studies. The antibody levels against MSP1a and MSP1b developed by vaccine preparations containing tick cell culture-derived A. marginale were very low, probably as a result of an overestimation of the amount of A. marginale antigen as discussed above.

Mapping of Bovine B-Bell Epitopes of MSP1a

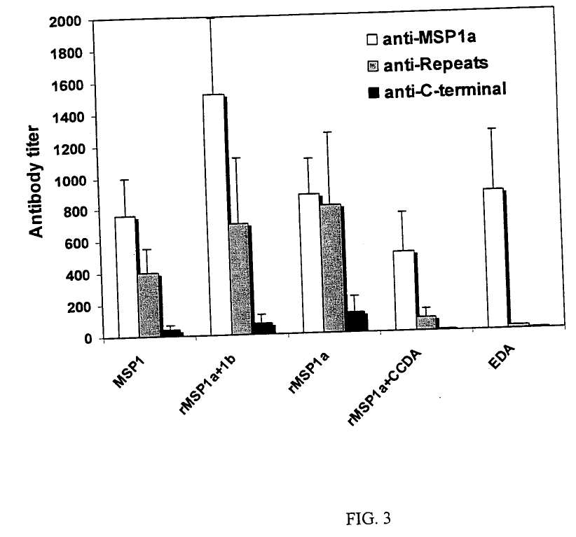

The antibody titers against MSP1a was higher in cattle vaccinated with immunogens containing rMSP1a or erythrocyte-derived A. marginale antigen (FIG. 2). This antibody response was primarily directed against the N-terminal repeated peptides of MSP1a (FIG. 3), indicating that this region contains immunodominant B-cell epitopes. In contrast, antibodies directed to the C-terminal region of MSP1a were detected at very low levels in all the immunization groups (FIG. 3), suggesting that bovine B-cell epitopes may not be present in the conserved region of MSP1a. In order to identify the epitopes recognized by sera from immunized cattle and to determine if recombinant and whole-organism vaccines elicit a response against the same or different B-cell epitopes of A. marginale MSP1a, sera from immunized cattle were reacted with synthetic peptides that spanned the entire sequence of MSP1a. Sera from groups of cattle immunized with rMSP1a, erythrocyte-derived A. marginale, tick cell culture-derived A. marginale, or cell culture-derived A. marginale plus rMSP1a, were pooled and used in this experiment. The four pools of sera recognized the same two peptides in the N-terminal repeats of MSP1a (FIG. 4). These two peptides share the sequence SSAGGQQQESS (SEQ. ID NO: 77) that is likely to contain the linear epitope recognized by the pools of bovine antisera. The epitopes recognized by these sera were the same regardless of the immunogen used. Interestingly, the pattern of response was very homogenous despite the fact that the experiments were carried out using mixed breed cattle.

Linear B-Cell Epitopes Recognized by Mouse and Rabbit Antibodies

Pooled sera from groups of Balb/c mice immunized with erythrocyte- or culture-derived A. marginale supplemented or not with rMSP1a recognized a set of epitopes different from those recognized by bovine sera (FIG. 4), but the epitopes were the same among immunization groups. None of the peptides recognized by mouse sera were located in the repeated peptides of MSP1a. Therefore, the mouse neutralization-sensitive epitope reported previously was not recognized by sera from immunized mice. Serum from a rabbit immunized with denatured rMSP1a antigen reacted with three peptides, only one of which was in the N-terminal repeats of MSP1a (FIG. 4). Another rabbit immunized with a synthetic peptide that models the N-terminal repeats recognized four consecutive peptides spanning a single repeat. Monoclonal antibody ANA15D₂, used as a positive control in the epitope mapping experiment, recognized two peptides in the N-terminal repeats that contained the reported sequence (QASTSS, SEQ. ID NO: 78) of the neutralization-sensitive epitope. All the epitopes recognized by bovine, rabbit and mouse antisera were predicted to be exposed on the surface of the outer membrane of A. marginale using the TMHMM2 algorithm (FIG. 4).

Protection against A. marginale Infection

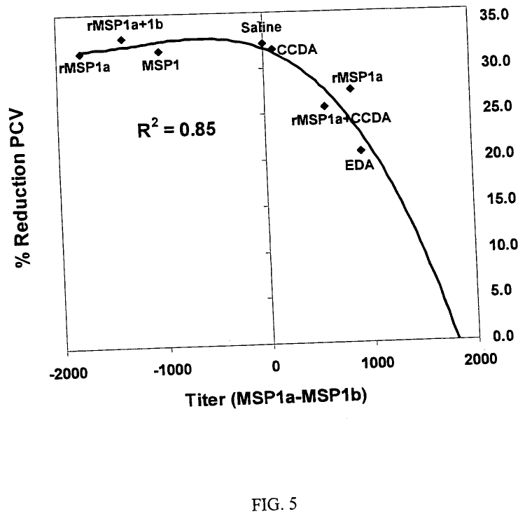

Immunized and control cattle were challenge-exposed with Virginia isolate A. marginale two weeks after the last immunization. All control animals, immunized with saline and adjuvant alone, developed signs of infection, with PPE ranging from 2.7% to 7.0% and an average 33% reduction PCV. The reduction in PCV was monitored as a measure of protection against heterologous A. marginale challenge and was significantly lower (p<0.05) in cattle with preferential anti-MSP1a response as compared to control animals or cattle with a preferential response against MSP1b (Table 2). However, the average percent reduction PCV did not correlate with the mean antibody titers against MSP1a or MSP1b (data not shown). The percent reduction in PCV of animals that developed a preferential response to MSP1b was not significantly different from that of control cattle. In addition, the mean differential of the antibody response against MSP1a and MSP1b (anti-MSP1a titer minus anti-MSP1b titer) correlated with the average percent reduction of PCV (FIG. 5). This correlation was accurately modeled by the fitted curve of a non-linear (third degree polynomial) equation. The highest degree of protection (lowest PCV reduction) was obtained in cattle immunized with erythrocyte-derived A. marginale antigen, followed by the group of cattle immunized with cell culture-derived A. marginale plus rMSP1a antigen (FIG. 5). Percent reduction PCV after heterologous challenge in animals vaccinated with immunogens that contained rMSP1b protein was not significantly different from percent reduction PCV in control cattle.

Discussion

The A. marginale/tick cell culture system provided an alternative source of A. marginale antigen for serologic tests and vaccine development. The efficacy of a vaccine preparation based on A. marginale derived from this tick cell culture system has been reported recently (Kocan et al., 2001; de la Fuente et al., 2002a). The MSPs of A. marginale derived from cultured tick cells and infected bovine erythrocytes have been shown to be structurally conserved (Barbet et al., 1999), but MSP1a was recently shown to be differentially expressed in A. marginale derived from bovine erythrocytes and tick cells (Garcia-Garcia et al., 2003). As a result, cattle immunized with erythrocyte-derived A. marginale develop a preferential response to MSP1a, whereas cattle immunized with tick cell culture-derived A. marginale respond preferentially to MSP1b (Kocan et al., 2001; de la Fuente et al., 2002a; Garcia-Garcia et al., 2003). Antibodies against A. marginale MSPs have been shown to be involved in different mechanisms of immune protection (reviewed by Palmer et al., 1999 and Kocan et al., 2003). MSP1a-specific antibodies appear to be particularly important in the inhibition of A. marginale adhesion to and invasion of cultured tick cells, tick salivary glands and bovine erythrocytes (McGarey et al., 1994; Blouin et al., 2003; de la Fuente et al., 2003a; 2003b). Therefore, the antibody response to A. marginale MSP1a elicited by recombinant and whole-organism vaccine preparations was characterized. Consistent with previous observations, the antibody response to MSP1a was higher in cattle immunized with erythrocyte-derived A. marginale or with vaccine preparations that contained rMSP1a protein. Moreover, the antibody response in these groups of cattle was directed primarily against the MSP1a repeats, and very low level of antibodies were detected against the conserved C-terminal region. Both the MSP1a repeat and C-terminal regions of MSP1a were previously reported to contain B-cell epitopes and similar levels of antibodies were reported to be elicited against each region in response to immunization of cattle with purified native MSP1 complex (Brown et al., 2001). However, the present results suggest that most of the bovine B-cell epitopes of MSP1a are located in the N-terminal hydrophilic region that contains the repeated peptides. Although the discrepancy might be due to the use of different antigen preparations, it is likely that differences are also due to variation in the immunoassays used to measure antibody levels. In this study we used quantitative immunoassays and all the antigens used for coating the ELISA plates were recombinant proteins expressed in E. coli. In the previous study, semiquantitative immunoblotting and dot blot assays, using synthetic versus recombinant antigens, respectively, were used for antibody quantification (Brown et al., 2001). The data reported here suggest that immunodominant bovine B-cell epitopes are located in the N-terminal repeats of MSP1a, which has previously shown to be necessary and sufficient for adhesion to bovine erythrocytes and tick cells (de la Fuente et al., 2003b). Antibodies against these repeats were also shown to inhibit binding to and infection of erythrocytes (Palmer et al., 1986; McGarey et al., 1994; de la Fuente et al., 2003b) and tick cells (Blouin et al., 2003; de la Fuente et al., 2003b). Therefore, the development of a strong MSP1a repeat specific antibody response may contribute to a protective response against A. marginale infection.

A neutralization-sensitive epitope recognized by mouse monoclonal antibody ANA15D₂ has been mapped to the repeated peptides of MSP1a (Allred et al., 1990). Although the presence of B-cell epitopes in the MSP1a repeats and C-terminal had been suggested (Brown et al., 2001), these epitopes have not been characterized previously. Using overlapping synthetic peptides covering the entire MSP1a sequence, linear B-cell epitopes recognized by pooled sera from cattle immunized with rMSP1a, or A. marginale derived from infected bovine erythrocytes or cultured tick cells were mapped. Only two peptides, located in the N-terminal repeats of MSP1a and containing the sequence SSAGGQQQESS (SEQ. ID NO: 77), were recognized by the four different pools of sera. These sera likely recognized the same linear B-cell epitope represented twice in the tandemly repeated peptides of the Oklahoma isolate MSP1a. This result is consistent with the observation that the main antibody response in immunized cattle was directed against the repeated peptides and not the C-terminal region of MSP1a. Moreover, CD4⁺ T-lymphocyte epitopes have been identified in the hydrophilic N-terminal region of MSP1a that contains the repeated peptides (Brown et al., 2002). Collectively, these results indicate that this region contains the T- and B-cell epitopes necessary for developing a protective immune response, and suggests the utility of the hydrophilic N-terminal portion of MSP1a for immunization and assessment of its protective capacity.

When sera from immunized mice and rabbits were used for epitope mapping, the linear epitopes recognized were different from the bovine B-cell epitopes described above, suggesting MHC-restriction or at least species-specificity of the B-cell epitopes of MSP1a. However, the same peptides were recognized by sera from all the immunization groups, suggesting that the B-cell epitopes of MSP1a are the same in the recombinant and whole organism vaccine preparations included in this study. Interestingly, immunized Balb/c mice did not develop antibodies against the linear mouse neutralization-sensitive epitope recognized by mAb ANA15D₂. This monoclonal antibody was likely obtained in a different mouse strain and, as discussed above, there may be MHC-restriction of the B-cell epitopes of MSP1a. The peptides containing the neutralizing epitope recognized by mAb ANA15D₂ did not react with bovine sera from any immunization group. A rabbit serum, known to inhibit adhesion of MSP1a to tick cells in an in vitro assay (de la Fuente et al., 2003b), reacted with four consecutive peptides that covered the whole sequence of a single repeat. Since the 16-mer peptides used for epitope mapping were synthesized with 8 amino acid overlaps, at least two different epitopes were recognized by this rabbit serum. The peptides recognized by this rabbit serum overlap with one of the peptides recognized by antisera from vaccinated cattle, but whether the antibodies react with the same or different epitope sequences is not known. In other experiments, sera from immunized cattle inhibited infection of tick cells by A. marginale (Blouin et al., 2003). All the linear epitopes identified in this study were predicted to be surface exposed by the TMHMM2 algorithm. These data validates the topology predicted for MSP1a in which only four transmembrane helices are present, in contrast to other models that predict five transmembrane domains in MSP1a (de la Fuente et al., 2001c).

Although all the immunogens tested produced an antibody response against the same two linear B-cell epitopes in immunized cattle, a significant difference in the average percent reduction PCV was observed among immunization groups. These results suggest that differences in protective efficacy might not be due to the development of an antibody response against different linear B-cell epitopes but rather due to differences in the amount of antibodies generated against these linear epitopes or other conformational epitopes. However, the antibody titers against MSP1a or MSP1b did not correlate with the level of protection against A. marginale infection. Since MSP1a is an integral membrane protein covalently linked to MSP1b (Vidotto et al., 1994), some of the B-cell epitopes of MSP1a may be involved in interactions with other molecules in the A. marginale membrane and therefore not be accessible for the development of an effective antibody response when cattle are immunized with whole organism preparations. Alternatively, overexpression of MSP1a in erythrocytic stages of A. marginale (Garcia-Gacia et al., 2003) may prevent antibodies from complete neutralization.

In an attempt to study the effect of the presence of MSP1b on the protective response mediated by MSP1a antibodies, a correlation analysis between the differential of the anti-MSP1a minus anti-MSP1b antibody titers and the protection against A. marginale infection as determined by the percent reduction PCV was performed. The fact that protection against heterologous challenge in cattle with a preferential antibody response against MSP1a was significantly higher than in cattle that developed a preferential response against MSP1b may indicate that MSP1a-specific antibodies are involved in the protective response against A. marginale infection, but the presence of other immunodominant proteins interacting with MSP1a, including MSP1b, may make inaccessible or alter the conformation of specific B-cell epitopes of MSP1a that are necessary for protection. Alternatively, some components of the whole A. marginale organism preparations may suppress the immune response against B-cell epitopes of MSP1a. Recent studies have suggested immune suppression by infection with A. marginale as a potential mechanism for persistence. A. marginale has been shown to be able to modulate the bovine immune system and suppress the response to certain epitopes during infection (Abbott, J. R., Palmer, G. H., Brown, W. C., 2003. 18th Meeting Am. Soc. Rickettsiol., Abstract No. 21). Even if the same epitopes were recognized and similar antibody levels were produced, the quality of the immune response and therefore the protective immunity could be affected by components of the whole organism vaccine preparations. A high titer of IgG2 antibodies, the opsonizing bovine IgG subclass, directed to surface exposed epitopes of A. marginale, has been associated with a protective immune response (Brown et al., 1998). Cattle with a mixed IgG1 and IgG2 response against specific MSPs are only partially protected against A. marginale challenge. Whether the antibody response to MSP1a is affected by these mechanisms of immunomodulation remains unknown. In addition, there might be differences in the CD4⁺ T-lymphocyte epitopes in the recombinant and whole organism vaccines evaluated in this study. A CD4+T-lymphocyte response has been shown to be involved in the development of protection against A. marginale infection (Brown et al., 1998; 2001; 2002).

Although the main goal of research directed to the present invention was to characterize the linear B-cell epitopes of MSP1a, conformational or non-peptidic epitopes of MSP1a might also be involved in protection against A. marginale infection. MSP1 a was recently shown to be glycosylated (Garcia-Garcia, J. C., de la Fuente, J., Bell, G., Blouin, E. F., Kocan, K. M., submitted for publication), and these carbohydrate modifications were suggested to play a role in adhesion of A. marginale to tick cells. Therefore, an antibody response against the glycans of MSP1a may also contribute to the neutralization of the function of MSP1a as an adhesin for host cells.

Collectively the results of this research suggest that immunization of cattle with A. marginale antigens that elicit a strong and preferential antibody response against MSP1a induce protection in vaccinated cattle. Since other A. marginale antigens may have a synergistic effect on protection, a vaccine preparation that contains whole A. marginale organisms supplemented with rMSP1a might induce a protective immune response mediated by antibodies against MSP1a as well as other protective antigens. Because antibodies to MSP1a have been shown to inhibit the infection of tick cells and bovine erythrocytes by A. marginale, the development of a vaccine based on a combination of rMSP1a with whole A. marginale organisms derived from tick cell culture to include the contribution of other antigens, might provide protection against bovine anaplasmosis and its transmission by the tick vector.

The preparation of pharmaceutical compositions (vaccines) utilizing the aforedescribed B-cell epitopes as distinct antigenic components is easily accomplished using well known methods and techniques. The vaccine and/or antigen preparation is combined into a formulation in an amount effective to provide for a protective immune response against infection with A. marginale. A protective immune response against A. marginale decreases the clinical signs of anaplasmosis. Clinical symptoms of anaplasmosis include a reduction in packed red cell volume of about 25 to 80% and parasitemia of the red blood cells of about 15 to 70%. A decrease in the symptoms of anaplasmosis includes prevention of the reduction in the packed red cell volume and a decrease in percent parasitemia. Preferably, a protective response includes packed red cell volume change of 25% or less compared with control animals and/or a decrease in parasitemia to about 5 to 25% of the red blood cells or less depending on the conditions. Measurements of packed red cell volume and percent parasitemia are conducted using standard methods. Vaccine preparations are combined with physiologically acceptable carriers to form vaccines. The preferred physiologically acceptable carrier is an oil-based adjuvant.

Preferably, the inventive formulation is set to contain about 100 micrograms of antigen in an oil-based adjuvant such as Xtend® III (Grand Laboratories, Larchwood, Iowa).

The vaccines may be administered by a variety of routes including intravenously, intraperitoneally, intramuscularly, and subcutaneously. The preferred route of administration is subcutaneous. The vaccine can be administered in a single dose or multiple doses until the desired effect is achieved.

While the invention has been described with a certain degree of particularity, it is understood that the invention is not limited to the embodiment(s) set for herein for purposes of exemplification, but is to be limited only by the scope of the attached claim or claims, including the full range of equivalency to which each element thereof is entitled.

References, all of which are incorporated herein by reference.
1. Allred, D. R., McGuire, T. C., Palmer, G. H., Leib, S. R., Harkins, T. M., McElwain, T. F., Barbet, A. F., 1990. Molecular basis for surface antigen size polymorphisms and conservation of a neutralization-sensitive epitope in Anaplasma marginale. Proc. Natl. Acad. Sci. 87, 3220-3224.
2. Arulkanthan, A., Brown, W. C., McGuire, T. C., Knowles, D. P., 1999. Biased immunoglobulin G1 isotype responses induced in cattle with DNA expressing msp1a of Anaplasma marginale. Infect. Immun. 67, 3481-3487.
3. Barbet, A. F., Blentlinger, R., Lundgren, A. M., Blouin, E. F., Kocan, K. M., 1999. Comparison of surface proteins of Anaplasma marginale grown in tick cell culture, tick salivary glands and cattle. Infect. Immun. 67, 102-107.
4. Blouin, E. F., Barbet, A. F., Jooyoung Yi, Kocan, K. M., Saliki, J. T., 2000. Establishment and characterization of an Oklahoma isolate of Anaplasma marginale in cultured Ixodes scapularis cells. Vet. Parasitol. 87, 301-313.
5. Blouin, E. F., Saliki, J. T., de la Fuente, J., Garcia-Garcia, J. C., Kocan, K. M., 2003. Antibodies to Anaplasma marginale major surface proteins 1a and 1b inhibit infectivity for cultured tick cells. Vet. Parasitol. 111, 247-260.
6. Brown, W. C., Shkap, V., Zhu, D., McGuire, T. C., Tuo, W., McElwain, T. F., Palmer, G. H., 1998. CD4⁺ T-lymphocyte and immunoglobulin G2 responses in calves immunized with Anaplasma marginale outer membranes and protected against homologous challenge. Infect. Immun. 66, 5406-5413.
7. Brown, W. C., Palmer, G. H., Lewin, H. A., McGuire, T. C., 2001. CD4(+) T lymphocytes from calves immunized with Anaplasma marginale major surface protein 1 (MSP1), a heteromeric complex of MSP1a and MSP1b, preferentially recognize the MSP1a carboxyl terminus that is conserved among strains. Infect. Immun. 69, 6853-6862.
8. Brown, W. C., McGuire, T. C., Mwangi, W., Kegerreis, K. A., Macmillan, H., Lewin, H. A., Palmer, G. H., 2002. Major histocompatibility complex class II DR-restricted memory CD4(+) T lymphocytes recognize conserved immunodominant epitopes of Anaplasma marginale major surface protein la. Infect. Immun. 70, 5521-5532.
9. de la Fuente, J., Garcia-Garcia, J. C., Blouin, E. F., Kocan, K. M., 2001a. Differential adhesion of major surface proteins 1a and 1b of the ehrlichial cattle pathogen Anaplasma marginale to bovine erythrocytes and tick cells. Int. J. Parasitol. 31, 145-153.
10. de la Fuente, J., Garcia-Garcia, J. C., Blouin, E. F., Kocan, K. M., 2001b. Major surface protein 1a effects tick infection and transmission of the ehrlichial pathogen Anaplasma marginale. Int. J. Parasitol. 31, 1705-1714.
11. de la Fuente, J., Garcia-Garcia, J. C., Blouin, E. F., Rodríguez, S. D., García, M. A., Kocan, K. M., 2001c. Evolution and function of tandem repeats in the major surface protein 1a of the ehrlichial pathogen Anaplasma marginale. Anim. Health Res. Rev. 2, 163-173.
12. de la Fuente, J., Kocan, K. M., Garcia-Garcia, J. C., Blouin, E. F., Claypool, P. L., Saliki, J. T., 2002a. Vaccination of cattle with Anaplasma marginale derived from tick cell culture and bovine erythrocytes followed by challenge-exposure with infected ticks. Vet. Microbiol. 89, 239-251.
13. de la Fuente, J., Garcia-Garcia, J. C., Blouin, E. F., Saliki, J. T., Kocan, K. M., 2002b. Infection of tick cells and bovine erythrocytes with one genotype of the intracellular ehrlichia Anaplasma marginale excludes infection with other genotypes. Clin. Diagn. Lab. Immunol. 9, 658-668.
14. de la Fuente, J., Garcia-Garcia, J. C., Blouin, E. F., Kocan, K. M., 2003b. Characterization of the functional domain of major surface protein la involved in adhesion of the rickettsia Anaplasma marginale to host cells. Vet. Microbiol. 91, 265-283.
15. de la Fuente, J., Kocan, K. M., Garcia-Garcia, J. C., Blouin, E. F., Halbur, T., Onet, V., 2003a. Antibodies to Anaplasma marginale major surface protein la reduce infectivity for ticks. The Journal of Applied Research in Veterinary Medicine, submitted.
16. Dikmans, G., 1950. The transmission of anaplasmosis. Am. J. Vet. Res. 11, 5-16. Ewing, S. A., 1981. Transmission of Anaplasma marginale by arthropods. In: Proceedings of the 7th National Anaplasmosis Conference, Mississippi State University, MS, USA, pp. 395-423.
17. Garcia-Garcia, J. C., de la Fuente, J., Blouin, E. F., Johnson, T. D., Halbur, T., Onet, V., Saliki, J. T., Kocan, K. M., 2003. Differential expression of the msp1a gene of Anaplasma marginale occurs in bovine erythrocytes and tick cells. Vet. Microbiol, in press.
18. Kocan, K. M, Halbur, T., Blouin, E. F., Onet, V., de la Fuente, J., Garcia-Garcia, J. C., Saliki, J. T., 2001. Immunization of cattle with Anaplasma marginale derived from tick cell culture. Vet. Parasitol. 102, 151-161.
19. Kocan, K. M, de la Fuente, J., Guglielmone, A. A., Meléndez, R. D., 2003. Antigens and alternatives for control of Anaplasma marginale infection in cattle. Clin. Microbiol. Rev. 16, 698-712.
20. Krogh, A., Larsson, B., von Heijne, G., Sonnhammer, E. L., 2001. Predicting transmembrane protein topology with a hidden Markov model: application to complete genomes. J. Mol. Biol. 305, 567-580.
21. Kuttler, K. L., 1984. Anaplasma infections in wild and domestic ruminants: a review. J. Wildl. Dis. 20, 12-20.
22. Laemmli, U. K., 1970. Cleavage of structural proteins during the assembly of the head of bacteriophage T4. Nature 227, 680-685.
23. McGarey, D. J., Allred, D. R., 1994. Characterization of hemagglutinating components on the Anaplasma marginale initial body surface and identification of possible adhesins. Infect. Immun. 62, 4587-4593.
24. McGarey, D. J., Barbet, A. F., Palmer, G. H., McGuire, T. C., Allred, D. R., 1994. Putative adhesins of Anaplasma marginale: major surface polypeptides 1a and 1b. Infect. Immun. 62, 4594-4601.
25. McGuire, T. C., Stephens, E. B., Palmer, G. H., McElwain, T. F., Leichtensteiger, C. A., Leib, S. R., Barbet, A. F., 1994. Recombinant vaccinia virus expression of Anaplasma marginale surface protein MSP-1a: effect of promoters, leader sequences and GPI anchor sequence on antibody response. Vaccine 12, 465-471.
26. Montenegro-James, S., James, M. A., Toro Benitez, M., Leon, E., Baek, B. K., Guillen, A. T., 1991. Efficacy of purified Anaplasma marginale initial bodies as a vaccine against anaplasmosis. Parasitol. Res. 77, 93-101.
27. Munderloh, U. G., Blouin, E. F., Kocan, K. M., Ge, N. L., Edwards, W. L., Kurtti, T. J., 1996. Establishment of the tick (Acari: Ixodidae)-borne cattle pathogen Anaplasma marginale (Rickettsiales: Anaplasmataceae) in tick cell culture. J. Med. Entomol. 33, 656-664.
28. Palmer, G. H., McGuire, T. C., 1984. Immune serum against Anaplasma marginale initial bodies neutralizes infectivity for cattle. J. Immunol. 133, 1010-1015.
29. Palmer, G. H., Barbet, A. F., Davis, W. C., McGuire, T. C., 1986. Immunization with an isolate-common surface protein protects cattle against anaplasmosis. Science 231, 1299-1302.
30. Palmer, G. H., Waghela, S. D., Barbet, A. F., Davis, W. C., McGuire, T. C., 1987. Characterization of a neutralization sensitive epitope on the AM 105 surface protein of Anaplasma marginale. Int. J. Parasitol. 17, 1279-1285.
31. Palmer, G. H., Oberle, S. M., Barbet, A. F., Goff, W. L., Davis, W. C., McGuire, T. C., 1988. Immunization of cattle with a 36-kilodalton surface protein induces protection against homologous and heterologous Anaplasma marginale challenge. Infect. Immun. 56, 1526-1531.
32. Palmer, G. H., Barbet, A. F., Cantor, G. H., McGuire, T. C., 1989. Immunization of cattle with the MSP-1 surface protein complex induces protection against a structurally variant Anaplasma marginale isolate. Infect. Immun. 57, 3666-3669.
33. Palmer, G. H., Rurangirwa, F. R., Kocan, K. M., Brown, W. C., 1999. Molecular basis for vaccine development against the ehrlichial pathogen Anaplasma marginale. Parasitol. Today 15, 281-286.
34. Saliki, J. T., Blouin, E. F., Rodgers, S. J., Kocan, K. M., 1998. Use of tick cell culture-derived Anaplasma marginale antigen in a competitive ELISA for serodiagnosis of anaplasmosis. Ann. N. Y. Acad. Sci. 849,273-281.
35. Tebele, N., McGuire, T. C., Palmer, G. H., 1991. Induction of protective immunity by using Anaplasma marginale initial body membranes. Infect. Immun. 59, 3199-3204.
36. Vidotto, M. C., McGuire, T. C., McElwain, T. F., Palmer, G. H., Knowles, D. P. Jr., 1994. Intermolecular relationships of major surface proteins of Anaplasma marginale. Infect. Immun. 62, 2940-2946.

37. Visser, E. S., McGuire, T. C., Palmer, G. H., Davis, W. C., Shkap, V., Pipano, E., Knowles, D. P. Jr., 1992. The Anaplasma marginale msp5 gene encodes a 19-kilodalton protein conserved in all recognized Anaplasma species. Infect. Immun. 60, 5139-5144.

TABLE 1
Immunization groups and immunogen composition.

| Immunization | |
|---|---|
| Group^{a} | Immunogen^{b} |
| rMSP1a + 1b | Recombinant MSP1a and MSP1b antigens |
| MSP1 | Recombinant MSP1 complex obtained in vitro |
| rMSP1a | Recombinant MSP1a antigen |
| rMSP1b | Recombinant MSP1b antigen |
| CCDA | Tick cell culture-derived A.marginale |
| rMSP1a + CCDA | Tick cell culture-derived A. marginale plus |
| | recombinant MSP1a |
| EDA | Erythrocyte-derived A.marginale |
| Saline | Adjuvant alone |

^{a}Only immunogens rMSP1a, rMSP1a + CCDA and EDA were used in the mouse immunization experiment. All eight groups were included in the cattle immunization experiment.
^{b}Doses of immunogen contained 10¹⁰ A. marginale organisms and/or 100 μg recombinant antigen in 5 ml oil adjuvant for cattle and 10⁸ A.marginale and/or 5 μg recombinant antigen in 100 μl for mice. Cattle were immunized at weeks 0, 4 and 7 and challenged at week 9 while mice were immunized at weeks 0 and 2 with blood collection at week 4.

TABLE 2
Percent reduction PCV and antibody response against MSP1a and MSP1b
in vaccinated cattle.

| Differential Titer^{a} | | Immunization | Reduction PCV^{b} |
|---|---|---|---|
| MSP1a − MSP1b | | Group | (%) |
| Negative | −1000 | MSP1 | 30.4 |
| | −1000 | MSP1 | 29.8 |
| | −500 | MSP1 | 34.7 |
| | −1500 | MSP1 | 30.8 |
| | −1000 | MSP1 | 34.5 |
| | −6000 | rMSP1a + 1b | 32.0 |
| | −1750 | rMSP1b | 32.7 |
| | −990 | rMSP1b | 36 |
| | −1750 | rMSP1b | 26.2 |
| | −3500 | rMSP1b | 36.0 |
| | −490 | rMSP1b | 28.7 |
| Mean ± SD | −1332 ± 704 | | 32.0 ± 3.2 |
| Positive | 500 | rMSP1a + 1b | 43.5 |
| | 1500 | rMSP1a + 1b | 34.8 |
| | 750 | rMSP1a | 23.5 |
| | 490 | rMSP1a | 30.7 |
| | 990 | rMSP1a | 38.1 |
| | 990 | rMSP1a | 18.0 |
| | 990 | rMSP1a | 27.0 |
| | 490 | CCDA | 22.4 |
| | 990 | rMSP1a + CCDA | 22.6 |
| | 240 | rMSP1a + CCDA | 34.7 |
| | 990 | rMSP1a + CCDA | 14.0 |
| | 240 | rMSP1a + CCDA | 29.2 |
| | 490 | rMSP1a + CCDA | 27.6 |
| | 490 | EDA | 6.9 |
| | 990 | EDA | 22.2 |
| | 750 | EDA | 27.5 |
| | 490 | EDA | 28.6 |
| | 1990 | EDA | 18.5 |
| Mean ± SD | 691 ± 299 | | 26.1 ± 8.8 |
| Neutral^{c} | | Saline | 33.0 |
| | | Saline | 26.2 |
| | | Saline | 42.8 |
| | | Saline | 24.6 |
| | | Saline | 36.4 |
| | | rMSP1a + 1b | 22.3 |
| | | rMSP1a + 1b | 34.6 |
| | | CCDA | 29.2 |
| | | CCDA | 43.5 |
| | | CCDA | 37.7 |
| | | CCDA | 27.4 |
| Mean ± SD | | | 32.5 ± 7.2 |

^{a}The differential titer was calculated subtracting the MSP1b antibody titer from the MSP1a antibody titer for each individual animal. Geometric mean antibody titers were calculated for each group of cattle.
^{b}The percent reduction PCV was calculated from the lowest PCV after heterologous A. marginale challenge-exposure and the PCV prior to challenge, % Reduction PCV = 100 × (1 − Initial PCV/Lowest PCV).
^{c}Control animals, immunized with saline and adjuvant only, and animals in which the antibody response against MSP1a and MSP1b was not biased (differential = 0), were grouped for this analysis.

1. A polypeptide comprising an isolated and purified B-cell epitope derived from the N-terminal repeated peptides of Anaplasma marginale major surface protein (MSP)1a.

2. The polypeptide according to claim 1 wherein said epitope is contained within the sequence of SEQ. ID NO: 77.

3. A pharmaceutical composition comprising at least one isolated and purified B-cell epitope derived from the N-terminal repeated peptides of Anaplasma marginale major surface protein (MSP)1a, wherein said composition further comprises a pharmaceutically acceptable carrier or diluent.

4. The composition according to claim 1 wherein said epitope is contained within the sequence of SEQ. ID NO: 77.

5. A method for inducing or enhancing an immune response in a ruminate against infection by A. marginale, said method comprising administering to said ruminant an effective amount of the pharmaceutical composition of claim 3.

6. The method according to claim 5, wherein said B-cell epitope is contained within the sequence of SEQ. ID NO: 77.


SEQUENCE LISTING (78 sequences)

SEQ ID NO: 1 - protein, 16 aa, Anaplasma marginale
MLAEYVSPQPADGSSA

SEQ ID NO: 2 - protein, 16 aa, Anaplasma marginale
QPADGSSAGGQQQESS

SEQ ID NO: 3 - protein, 16 aa, Anaplasma marginale
GGQQQESSVSSQSDQA

SEQ ID NO: 4 - protein, 16 aa, Anaplasma marginale
VSSQSDQASTSSQLGA

SEQ ID NO: 5 - protein, 16 aa, Anaplasma marginale
STSSQLGADSSSAGGQ

SEQ ID NO: 6 - protein, 16 aa, Anaplasma marginale
DSSSAGGQQQESSVSS

SEQ ID NO: 7 - protein, 16 aa, Anaplasma marginale
QQESSVSSQSGQASTS

SEQ ID NO: 8 - protein, 16 aa, Anaplasma marginale
QSGQASTSSQLGTDSS

SEQ ID NO: 9 - protein, 16 aa, Anaplasma marginale
SQLGTDSSSASGQQQE

SEQ ID NO: 10 - protein, 16 aa, Anaplasma marginale
SASGQQQESSVSSQSG

SEQ ID NO: 11 - protein, 16 aa, Anaplasma marginale
SSVSSQSGQASTSSQS

SEQ ID NO: 12 - protein, 16 aa, Anaplasma marginale
QASTSSQSGANWRQEM

SEQ ID NO: 13 - protein, 16 aa, Anaplasma marginale
GANWRQEMRSKVASVE

SEQ ID NO: 14 - protein, 16 aa, Anaplasma marginale
RSKVASVEYILAARAL

SEQ ID NO: 15 - protein, 16 aa, Anaplasma marginale
YILAARALISVGVYAA

SEQ ID NO: 16 - protein, 16 aa, Anaplasma marginale
ISVGVYAAQGEIAKSQ

SEQ ID NO: 17 - protein, 16 aa, Anaplasma marginale
QGEIAKSQGCAPLRVA

SEQ ID NO: 18 - protein, 16 aa, Anaplasma marginale
GCAPLRVAEVEEIVRD

SEQ ID NO: 19 - protein, 16 aa, Anaplasma marginale
EVEEIVRDGLVRSHFH

SEQ ID NO: 20 - protein, 16 aa, Anaplasma marginale
GLVRSHFHDSGLSLGS

SEQ ID NO: 21 - protein, 16 aa, Anaplasma marginale
DSGLSLGSIRLVLMQV

SEQ ID NO: 22 - protein, 16 aa, Anaplasma marginale
IRLVLMQVGDKLGLQG

SEQ ID NO: 23 - protein, 16 aa, Anaplasma marginale
GDKLGLQGLKIGEGYA

SEQ ID NO: 24 - protein, 16 aa, Anaplasma marginale
LKIGEGYATYLAQAFA

SEQ ID NO: 25 - protein, 16 aa, Anaplasma marginale
TYLAQAFADNVVVAAD

SEQ ID NO: 26 - protein, 16 aa, Anaplasma marginale
DNVVVAADVQSGGACS

SEQ ID NO: 27 - protein, 16 aa, Anaplasma marginale
VQSGGACSASLDSAIA

SEQ ID NO: 28 - protein, 16 aa, Anaplasma marginale
ASLDSAIANVETSWSL

SEQ ID NO: 29 - protein, 16 aa, Anaplasma marginale
NVETSWSLHGGLVSKD

SEQ ID NO: 30 - protein, 16 aa, Anaplasma marginale
HGGLVSKDFDRDTKVE

SEQ ID NO: 31 - protein, 16 aa, Anaplasma marginale
FDRDTKVERGDLEAFV

SEQ ID NO: 32 - protein, 16 aa, Anaplasma marginale
RGDLEAFVDFMFGGVS

SEQ ID NO: 33 - protein, 16 aa, Anaplasma marginale
DFMFGGVSYNDGNASA

SEQ ID NO: 34 - protein, 16 aa, Anaplasma marginale
YNDGNASAARSVLETL

SEQ ID NO: 35 - protein, 16 aa, Anaplasma marginale
ARSVLETLAGHVDALG

SEQ ID NO: 36 - protein, 16 aa, Anaplasma marginale
AGHVDALGISYNQLDK

SEQ ID NO: 37 - protein, 16 aa, Anaplasma marginale
ISYNQLDKLDADTLYS

SEQ ID NO: 38 - protein, 16 aa, Anaplasma marginale
LDADTLYSVVSFSAGS

SEQ ID NO: 39 - protein, 16 aa, Anaplasma marginale
VVSFSAGSAIDRGAVS

SEQ ID NO: 40 - protein, 16 aa, Anaplasma marginale
AIDRGAVSDAADKFRV

SEQ ID NO: 41 - protein, 16 aa, Anaplasma marginale
DAADKFRVMMFGGAPA

SEQ ID NO: 42 - protein, 16 aa, Anaplasma marginale
MMFGGAPAGQEKTAEP

SEQ ID NO: 43 - protein, 16 aa, Anaplasma marginale
GQEKTAEPEHEAATPS

SEQ ID NO: 44 - protein, 16 aa, Anaplasma marginale
EHEAATPSASSVPSTV

SEQ ID NO: 45 - protein, 16 aa, Anaplasma marginale
ASSVPSTVHGKVVDAV

SEQ ID NO: 46 - protein, 16 aa, Anaplasma marginale
HGKVVDAVDRAKEAAK

SEQ ID NO: 47 - protein, 16 aa, Anaplasma marginale
DRAKEAAKQAYAGVRK

SEQ ID NO: 48 - protein, 16 aa, Anaplasma marginale
QAYAGVRKRYVAKPSD

SEQ ID NO: 49 - protein, 16 aa, Anaplasma marginale
RYVAKPSDTTTQLVVA

SEQ ID NO: 50 - protein, 16 aa, Anaplasma marginale
TTTQLVVAITALLITA

SEQ ID NO: 51 - protein, 16 aa, Anaplasma marginale
ITALLITAFAICACLE

SEQ ID NO: 52 - protein, 16 aa, Anaplasma marginale
FAICACLEPRLIGASG

SEQ ID NO: 53 - protein, 16 aa, Anaplasma marginale
PRLIGASGPLIWGCLA

SEQ ID NO: 54 - protein, 16 aa, Anaplasma marginale
PLIWGCLALVALLPLL

SEQ ID NO: 55 - protein, 16 aa, Anaplasma marginale
LVALLPLLGMAVHTAV

SEQ ID NO: 56 - protein, 16 aa, Anaplasma marginale
GMAVHTAVSASSQKKA

SEQ ID NO: 57 - protein, 16 aa, Anaplasma marginale
SASSQKKAAGGAQRVA

SEQ ID NO: 58 - protein, 16 aa, Anaplasma marginale
AGGAQRVAAQERSREL

SEQ ID NO: 59 - protein, 16 aa, Anaplasma marginale
AQERSRELSRARQEDQ

SEQ ID NO: 60 - protein, 16 aa, Anaplasma marginale
SRARQEDQQKLHVPAI

SEQ ID NO: 61 - protein, 16 aa, Anaplasma marginale
QKLHVPAILTGLSVLV

SEQ ID NO: 62 - protein, 16 aa, Anaplasma marginale
LTGLSVLVFIAAVVAC

SEQ ID NO: 63 - protein, 16 aa, Anaplasma marginale
FIAAVVACIAVDARRG

SEQ ID NO: 64 - protein, 16 aa, Anaplasma marginale
IAVDARRGTWQGSICF

SEQ ID NO: 65 - protein, 16 aa, Anaplasma marginale
TWQGSICFLAAFVLFA

SEQ ID NO: 66 - protein, 16 aa, Anaplasma marginale
LAAFVLFAISAAVVMA

SEQ ID NO: 67 - protein, 16 aa, Anaplasma marginale
ISAAVVMATRDQSLAE

SEQ ID NO: 68 - protein, 16 aa, Anaplasma marginale
TRDQSLAEECDSKCAT

SEQ ID NO: 69 - protein, 16 aa, Anaplasma marginale
ECDSKCATARTAQAVP

SEQ ID NO: 70 - protein, 16 aa, Anaplasma marginale
ARTAQAVPGGQQQPRA

SEQ ID NO: 71 - protein, 16 aa, Anaplasma marginale
GGQQQPRATEGVVSGG

SEQ ID NO: 72 - protein, 16 aa, Anaplasma marginale
TEGVVSGGSQEGGAGV

SEQ ID NO: 73 - protein, 16 aa, Anaplasma marginale
SQEGGAGVPGTSVPSA

SEQ ID NO: 74 - protein, 16 aa, Anaplasma marginale
PGTSVPSAGSGSVPPA

SEQ ID NO: 75 - protein, 16 aa, Anaplasma marginale
GSGSVPPATIMVSVDP

SEQ ID NO: 76 - protein, 16 aa, Anaplasma marginale
TIMVSVDPQLVATLGA

SEQ ID NO: 77 - protein, 11 aa, Anaplasma marginale
SSAGGQQQESS

SEQ ID NO: 78 - protein, 6 aa, Anaplasma marginale
QASTSS